(19) United States
(12) Patent Application Publication
(10) Pub. No.: US 2016/0067193 A1
(43) Pub. Date: Mar. 10, 2016

(54) TOPICAL PTEROSTILBENE COMPOSITIONS FOR USE IN TREATING UV-INDUCED SKIN DAMAGE AND HYPERPLASIA

(71) Applicant: The Regents of the University of California, Oakland, CA (US)

(72) Inventors: Frank L. Meyskens, JR., Irvine, CA (US); Ryan W. Dellinger, Azusa, CA (US)

(21) Appl. No.: 14/845,171

(22) Filed: Sep. 3, 2015

Related U.S. Application Data
(60) Provisional application No. 62/046,065, filed on Sep. 4, 2014.

(51) Int. Cl. A61K 31/09; A61K 9/00

(52) U.S. Cl. CPC A61K 31/09; A61K 9/0014

(57) ABSTRACT

A chemoprotective method for treating, inhibiting or preventing DNA damage in skin and/or hyperplasia caused by ultraviolet (UV) light by using an effective amount of pterostilbene is provided. Pharmaceutical and nutraceutical compositions containing pterostilbene suitable for administration to an individual in order to prevent subsequent UV-mediated DNA damage and/or hyperplasia in skin are provided.

### CROSS-REFERENCE TO RELATED APPLICATIONS

This application claims the benefit of U.S. Provisional application No. 62/046,065, filed on Sep. 4, 2014, which is hereby incorporated by reference herein.

### STATEMENT REGARDING FEDERALLY SPONSORED RESEARCH

This invention was made with government support under Grant No. P30CA62330, awarded by the National Cancer Institute, and Grant No. R03ES019668, awarded by the National Institute of Environmental Health Sciences. The Government has certain rights in the invention.

### FIELD OF THE INVENTION

A chemoprotective method for treating, inhibiting or preventing DNA damage in skin and/or hyperplasia caused by ultraviolet (UV) light by using an effective amount of pterostilbene is described. Pharmaceutical and nutraceutical compositions containing pterostilbene suitable for administration to an individual in order to prevent UV-mediated DNA damage and/or hyperplasia in skin are described. Compositions containing pterostilbene may be used in the care or treatment of skin and skin conditions.

### BACKGROUND

Skin is the human body's first and best defense against environmental exposures including solar ultraviolet (UV) radiation. Exposure to UV light is a key factor in the development of skin disorders including cancer. Skin cancer is the most prevalent type of cancer in the United States, affecting an estimated one out of every seven Americans (Ndiaye, et al., Arch. Biochem. Biophys. (2011) 508: 164-70).

Nonmelanoma skin cancer (NMSC) has increased rapidly in the past two decades and more than one million new cases of non-melanoma skin cancer (NMSC) are diagnosed annually in the United States. It is suspected that this estimate is low as squamous cell carcinoma (SCC) and basal cell carcinoma (BCC) of the skin are not required to be reported and the number of actual cases annually is projected to be over 3 million new cases annually (Wheless, et al., “Nonmelanoma skin cancer and the risk of second primary cancers: a systematic review,” Cancer Epidemiol. Biomarkers Prev. (2010) 19: 1686-95). Based on IC9 Codes, it is estimated that 5% of the Medicare budget is used in the care of these patients (Rogers, et al., “Incidence estimate of nonmelanoma skin cancer in the United States, 2006,” Arch. Dermatol. (2010) 146: 283-7).

An estimated 2,700 deaths this year will be the result of NMSC in the US. The majority of these deaths are caused by SCC. Most NMSC, including SCC are caused by sun exposure (including UV-B light) with resultant photocarcinogenesis. Epidemiological data also shows an increased risk of other lethal cancer types in individuals with a history of skin cancer. Thus, it is vital to understand the harmful effects that UV light has on the skin so that effective methods of treatment or prevention can be developed.

Actinic keratoses (AKs) are precancerous cutaneous neoplasms, which can give rise to SCC. They arise as a result of long-term sun exposure. Other causes of AKs and NMSC are UV light from tanning booths or arc welding, x-irradiation, or exposure to certain chemicals. AKs are extremely common lesions and are present in more than 10 million Americans. In one sample population, the yearly rate of progression of an AK to a SCC in an average-risk person in Australia is between 8 and 24 per 10,000. High-risk individuals (those with multiple AKs) have progression rates as high as 12-30 percent over 3 years. Two percent of SCCs originating in AKs may metastasize, and 7 percent recur locally.

Actinic keratoses are treated most commonly with liquid nitrogen or a topical chemotherapeutic agent, such as, for example, 5-fluorouracil. Less commonly they are treated with other topical agents (diclofenac and imiquimod), photodynamic therapy, chemical peels or ablative laser resurfacing. Treatment for NMSC is usually surgical, often resulting in scarring and other morbidities.

In the instant specification, it will be understood that “actinic keratosis” is the proliferative disorder that produces AKs.

While some treatments are known, it would be desirable to proactively prevent or inhibit formation of actinic keratosis, hyperplasia, and/or skin cancers in order to reduce treatment costs, morbidity, and mortality. A lifetime of sun protection is an excellent method for minimizing risk of development of actinic keratosi, hyperplasia, and/or NMSC. However, a large percentage of patients already have extensive photodamage and changing sun-protective behaviors has proven to be difficult.

An ideal chemopreventive agent could achieve regression of precancerous changes, prevent development of NMSC and minimize ultraviolet light associated damage with minimal or no side effects. As noted above, there are topical agents that can remove actinic keratoses but they generally result in significant inflammation at the treatment site. A novel approach is required.

UV-Mediated DNA Damage

Ultraviolet (UV) light plays an integral role in the development of numerous skin ailments ranging from aging to cancer. Considerable evidence spanning decades has conclusively demonstrated that UV radiation triggers multiple independent cellular responses. UV radiation is known to penetrate skin where it is absorbed by proteins, lipids and DNA, causing a series of events that result in progressive deterioration of the cellular structure and function of cells (Valacchi, et al., “Cutaneous responses to environmental stressors,” Ann. N.Y. Acad. Sci. (2012) 1271: 75-81). DNA is the building block of life and its stability is of the utmost importance for the proper functioning of all living cells. UV radiation is one of the most powerful (and common) environmental factors that can cause a wide range of cellular disorders by inducing mutagenic and cytotoxic DNA lesions; most notably cyclobutane-pyrimidine dimers (CPDs) and 6-4 photoproducts (64 pps) (Narayanan, et al., “Ultraviolet radiation and skin cancer,” Int. J. Dermatol. (2010) 49: 978-86). It is important to note that UV-mediated DNA damage is an early event in a plethora of proliferative cellular disorders. The two major types of UV-induced DNA damage are CPDs and 64pp (along with their Dewer isomers) (Sinha, R. P. and Hader, D. P., “UV-induced DNA damage and repair: a review,” Photochem. Photobiol. Sci. (2002) 1: 225-36; and Rastogi, et al., “Molecular mechanisms of ultraviolet radiation-induced DNA damage and repair,” J. Nucleic Acids (2010) 2010: 592980). These abundant DNA lesions, if unrepaired, can interfere with DNA replication and subsequently cause mutations in DNA. Thus, these lesions can be mutagenic (potentially leading to proliferative disorders) and/or can be cytotoxic (resulting in cell death). 64 pp occur at about one third the frequency of CPDs, but are more mutagenic (Sinha & Hader, 2002). In one embodiment, prevention of these UV-mediated DNA adducts is paramount to guarding against the onset of several proliferative disorders, ranging from aging to cancer.

UV-Mediated Hyperplasia

Increased keratinocyte proliferation, resulting in hyperplasia, is another major detrimental effect caused by UV exposure. This thickening of the skin is a direct result of the body trying to protect itself after excessive exposure to UV light. However, epidermal hyperplasia also increases the risk of skin cancer (Bowden, G. T., “Prevention of non-melanoma skin cancer by targeting ultraviolet-B-light signalling,” Nat. Rev. Cancer (2004) 4: 23-35). In another embodiment, prevention of UV-mediated hyperplasia is paramount to guarding against the onset of several proliferative disorders, ranging from aging to cancer.

Resveratrol, a natural polyphenol present in grapes and red wine, exerts several beneficial effects including antioxidant, chemopreventative and cardioprotective (Park, K. and Lee, J-H., “Protective effects of resveratrol on UVB-irradiated HaCaT cells through attenuation of the caspase pathway,” Oncol. Rep. (2008) 19: 413-7). Several studies have shown that resveratrol prevents UV-B mediated cell damage (including hyperplasia) in mouse skin when given orally or applied topically (Afaq, F., et al., “Prevention of short-term ultraviolet B radiation-mediated damages by resveratrol in SKH-1 hairless mice,” Toxicol. Appl. Pharmacol. (2003) 186(1): 28-37; Reagan-Shaw, S., et al., “Modulations of critical cell cycle regulatory events during chemoprevention of ultraviolet B-mediated responses by resveratrol in SKH-1 hairless mouse skin,” Oncogene (2004) 23(30): 5151-60; Aziz, M. H., et al., “Prevention of ultraviolet-B radiation damage by resveratrol in mouse skin is mediated via modulation in survivin,” Photochem. Photobiol. (2005) 81(1): 25-31; and Kim, K. H., et al. “Resveratrol Targets Transforming Growth Factor-beta2 Signaling to Block UV-Induced Tumor Progression,” J. Invest. Dermatol. (2011) 131: 195-202). Resveratrol has been shown to block UV-induced skin cancer progression in several mouse studies (Athar, M., et al., “Resveratrol: a review of preclinical studies for human cancer prevention,” Toxicol. Appl. Pharmacol. (2007) 224: 274-83). However, its use in humans as a chemopreventative agent seems to be unlikely (at least as a single agent) due to poor bioavailability (Roupe, K. A., et al. “Pharmacometrics of stilbenes: seguing towards the clinic,” Curr. Clin. Pharmacol. (2006) 1: 81-101). Resveratrol is well tolerated in humans, but is readily metabolized (by the UGTs) leading to a short half-life which hinders its effectiveness as a chemopreventative agent (Cottart C. H., et al., “Resveratrol bioavailability and toxicity in humans,” Mol. Nutr. Food Res. (2010) 54: 7-16).

### SUMMARY

A skin care composition includes pterostilbene for treating, inhibiting or preventing UV-mediated DNA damage and/or hyperplasia in human skin.

A method of treating, inhibiting or preventing UV-mediated DNA damage and/or hyperplasia in human skin is provided, comprising administering to the individual in need of such treatment an effective amount of the compound pterostilbene (e.g., by topical administration).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts metabolism of resveratrol and pterostilbene. Human liver microsomes metabolize resveratrol much faster than pterostilbene. In vitro glucuronidation assays are shown examining the ability of 100 μg of human liver microsomes to glucuronidate either 500 μM resveratrol (RES; upper panel) or 500 μM pterostilbene (PTERO; lower panel) in 2 hours. HPLC chromatographs are presented depicting either parent compound or indicated glucuronide metabolite. Glucuronide metabolite peaks were confirmed by subsequent treatment with β-glucuronidase (data not shown). Detection of peaks by UV/VIS detector at 308 nm.

FIG. 2 depicts the manner in which pterostilbene prevents UV-B induced damage in the skin of SKH-1 mice. Mice were treated topically with vehicle alone (‘+’ acetone), resveratrol or pterostilbene 30 minutes prior to exposure to 180 mJ/cm² of UV-B. Treatment was repeated every other day for 14 days. Photographs show representative mice after 4th treatment.

FIGS. 3A and 3B depict the manner in which pterostilbene prevents UV-B induced DNA damage in the skin of SKH-1 mice, namely (A) CPD or (B) 64 pp DNA damage. ELISAs are shown for different arms of mouse study (Abs. 492 nm). * indicates p<0.01 as compared with vehicle (VEH). (RES) is resveratrol and (PTERO) is pterostilbene. NS is not significant.

FIG. 3C depicts the manner in which pterostilbene prevents UV-mediated bi-fold skin thickening. Bi-fold skin measurements (mm) were made using a digital caliper. At least three measurements for each mouse were taken along the back. VEH is vehicle, RES is resveratrol, and PTERO is pterostilbene. *indicates p<0.01 as compared to control.

FIG. 4 depicts the manner in which pterostilbene prevents UV-mediated hyperplasia. H&E stains of back skins from mice in the indicated treatment arms (i.e., UV-B treated and control) were performed.

### DETAILED DESCRIPTION

A chemoprotective method for treating, inhibiting or preventing DNA damage in skin and/or hyperplasia caused by ultraviolet (UV) light by using an effective amount of pterostilbene has been discovered. Pharmaceutical and nutraceutical compositions containing pterostilbene suitable for administration to an individual in order to prevent subsequent UV-mediated DNA damage in skin are described.

The term “effective amount” is used herein to refer to an amount of a pterostilbene that is sufficient to treat, inhibit or prevent UV-mediated DNA damage and/or hyperplasia to any degree, e.g., as measured by any means described herein or known in the art. An effective amount for treatment (i.e., a therapeutically effective amount) is amount that typically produces any improvement in a condition, such as DNA damage or hyperplasia, relative to that condition before initiating treatment. “Inhibition” or “prevention” can be determined relative to the results observed in a control individual that is not treated with pterostilbene. The control individual is one that is matched to a treated individual as is standard in the art (e.g., as illustrated in the Examples described below. In various embodiments, an effective amount in an amount sufficient to treat, inhibit or prevent UV- mediated DNA damage and/or hyperplasia by at least: 10%, 20%, 30%, 40%, 50%, 60%, 70%, 80%, 90%, or 100%, as compared to a control. An effective amount is typically determined before the chemoprotective methods described herein are carried out.

In some embodiments, “an individual in need of treatment for, inhibition of, or prevention of, UV-mediated DNA damage or hyperplasia” excludes individuals who have been treated with pterostilbene for a different purpose.

Pterostilbene (3,5-dimethoxy-4′-hydroxy-trans-stilbene) is an orally bioavailable compound with a half life t_{1/2} of about 105 minutes in blood. In contrast, resveratrol has poor bioavailability, and is readily metabolized by UGTs leading to a much shorter half life (t_{1/2} about 14 minutes in blood), which hinders its effectiveness as a chemopreventive agent.

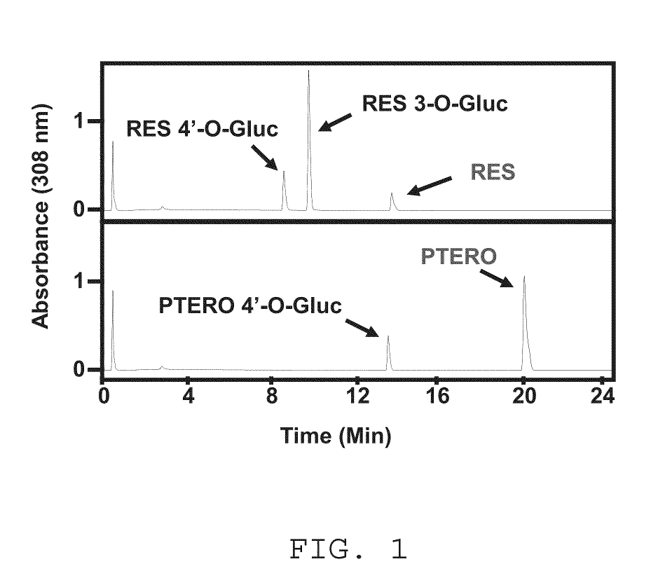

Pterostilbene is a natural product found in grapes and berries. Chemically it is a naturally occurring dimethylated analog of resveratrol, but has a longer half-life (FIG. 1) most likely due to the methyl groups dramatically reducing its metabolism by UGTs (Hougee, S., et al., “Selective COX-2 inhibition by a Pterocarpus marsupium extract characterized by pterostilbene, and its activity in healthy human volunteers,” Planta Med. (2005) 71: 387-92). 2005) 71(5): 387-92). Pterostilbene has been shown to be equally or significantly more potent than resveratrol in several biological assays in mice including inhibition of NF-KB, AP-1 and iNOS activation in mouse skin. Importantly, pterostilbene has been shown to prevent COX-2 activation and 12-O-tetradecanoylphorbol-13-acetate (TPA)-induced tumor formation in mouse epidermis in vivo (Cichocki, M., et al., “Pterostilbene is equally potent as resveratrol in inhibiting 12-O-tetradecanoylphorbol-13-acetate activated NFkappaB, AP-1, COX-2, and iNOS in mouse epidermis,” Mol. Nutr. Food Res. (2008) 52 Suppl 1: S62-70).

Herein is described a process using pterostilbene to inhibit, treat or prevent UV mediated DNA damage in skin, an early event in cellular proliferative disorders and the main direct cause of cellular damage by ultraviolet light. Also described is a process using pterostilbene to inhibit, treat or prevent UV mediated hyperplasia in skin, an early event in cellular proliferative disorders. Hyperplasia increases an individual's risk of skin cancer.

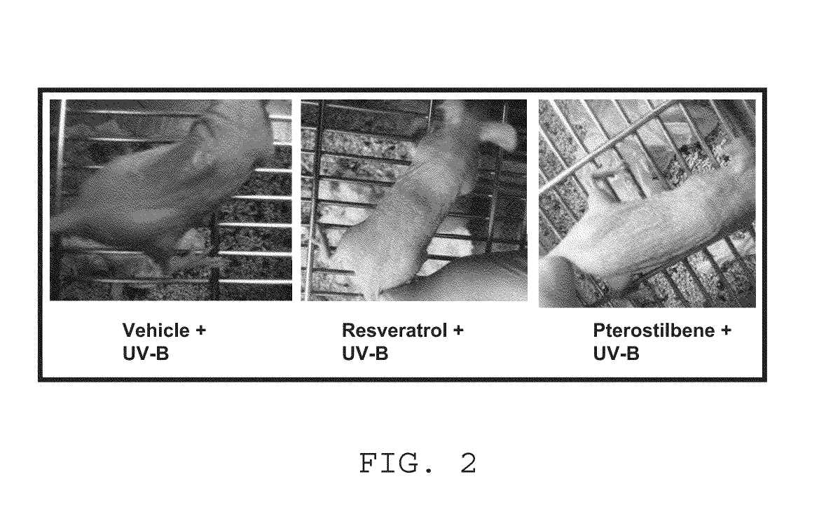

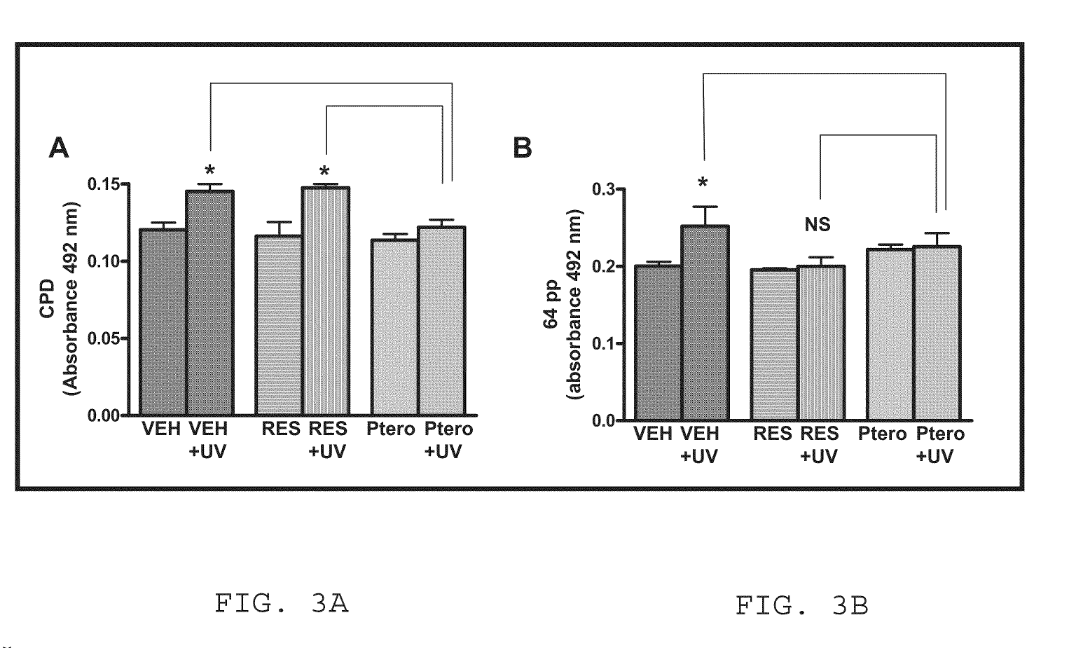

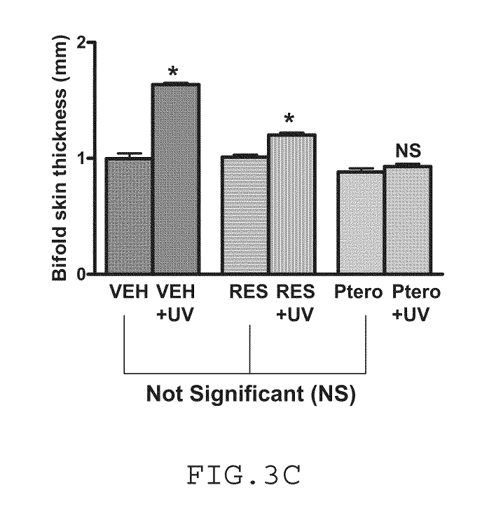

In certain embodiments, pterostilbene exhibits chemoprotective characteristics by preventing UV induced DNA damage in mouse skin (FIG. 2). It has been shown herein that pterostilbene prevents both CPD and 64 pp formation after UV exposure. Interestingly, resveratrol only prevented 64 pp formation and not CPD formation (FIGS. 3A and 3B). Thus, it is believed that pterostilbene will be effective in the prevention of numerous UV-mediated cellular proliferative disorders of human skin.

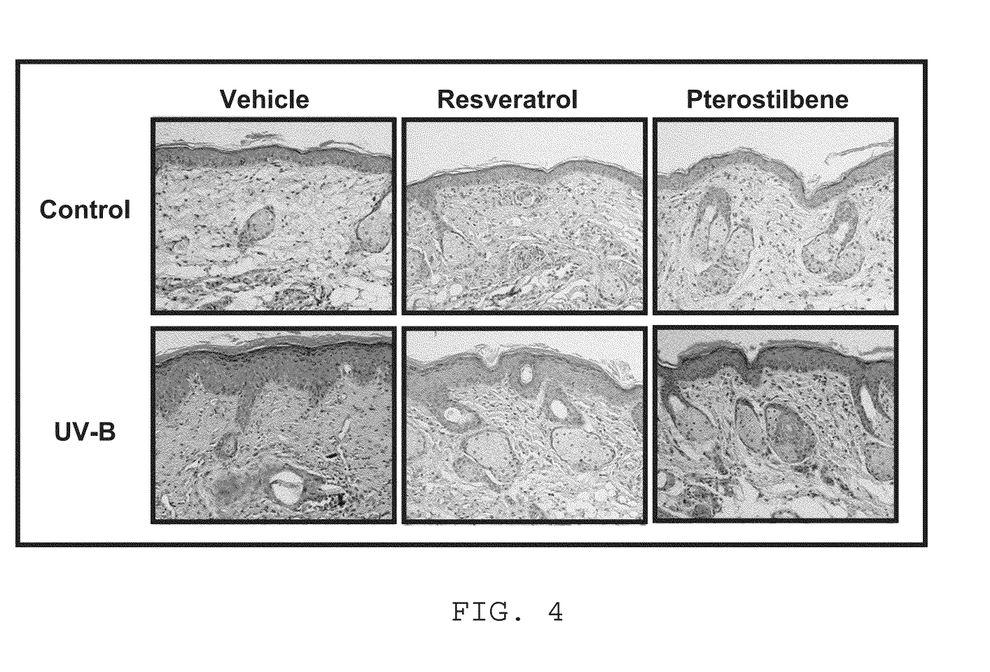

In an alternative embodiment, pterostilbene exhibits chemoprotective characteristics by preventing UV induced hyperplasia in mouse skin (FIGS. 2, 3C, and 4). Specifically, we discovered that pterostilbene prevented both bi-fold skin thickening (FIG. 3C) and hyperplasia (FIG. 4) after UV exposure. Interestingly, pterostilbene was more effective than resveratrol in all cases (FIGS. 2, 3C, and 4). Thus, it is believed that pterostilbene will be effective in the prevention of numerous UV-mediated cellular proliferative disorders of human skin.

Pterostilbene received GRAS (generally recognized as safe) certification in May 2011 and has been a commercially available nutraceutical marketed as a dietary supplement in pill form ever since. Clinically, pterostilbene went “first in humans” in December 2010 at the University of Mississippi with cholesterol, blood pressure and oxidative stress as endpoints (clinicaltrials.gov identifier number NCT01267227). In this study, a reduction in overall blood pressure was observed along with no toxicity reported in patients taking 125 mg of pterostilbene orally twice a day (Riche, D. M., et al., “Analysis of safety from a human clinical trial with pterostilbene,” J. Toxicol. (2013) 2013: 463595). Thus, pterostilbene is an ideal agent to prevent UV-mediated skin damage based on its human safety profile.

(Example A) Pterostilbene is more bioavailable than resveratrol in vitro.

Using pooled human liver microsomes (n=20) it was demonstrated for the first time that resveratrol is metabolized much faster than pterostilbene (See, FIG. 1). Specifically, in vitro glucuronidation assays were performed as previously described in one co-inventor's work (Dellinger, R. W., et al., “Glucuronidation of PhIP and N—OH-PhIP by UDP-glucuronosyltransferase 1A10,” Carcinogenesis (2007) 28: 2412-8; Dellinger, R. W., et al., “Importance of UDP-glucuronosyltransferase 1A10 (UGT1A10) in the detoxification of polycyclic aromatic hydrocarbons: decreased glucuronidative activity of the UGT1A10139Lys isoform,” Drug Metab. Dispos. (2006) 34: 943-9; Chen, G., Dellinger, R. W., et al., “Glucuronidation of tobacco-specific nitrosamines by UGT2B10,” Drug Metab. Dispos. (2008) 36: 824-30) using 100 μg of pooled human liver microsomes to assay the relative rates of glucuronidation of resveratrol vs. pterostilbene. After 2 hours, resveratrol was almost completely converted to its glucuronides (FIG. 1; upper panel), while pterostilbene is only 20% metabolized (FIG. 1; lower panel) indicating that pterostilbene will have a significantly higher half-life in humans. Consistent with previous reports, the 3-OH position of resveratrol is the preferred site of glucuronidation, while that position is methylated (and thus cannot be glucuronidated) in pterostilbene (Aumont, V., et al., “Regioselective and stereospecific glucuronidation of trans- and cis-resveratrol in human,” Arch. Biochem. Biophys. (2001) 393: 281-9).

Furthermore, increased bioavailability of pterostilbene has been demonstrated conclusively in rats, where pterostilbene showed 5-fold increased bioavailability (Kapetanovic, I. M., et al., “Pharmacokinetics, oral bioavailability, and metabolic profile of resveratrol and its dimethylether analog, pterostilbene, in rats,” Cancer Chemother. Pharmacol. (2011) 68: 593-601).

The overall rationale for studying nonmelanoma skin cancer (NMSC), particularly squamous cell carcinoma (SCC) and its precursors (including DNA damage of skin cells), emanates from its high and increasing frequency as the population ages, relative ease of accessibility of human tissue, increasing morbidity and mortality in immunocompromised individuals; SCC of the skin also serves as a potential model for understanding the biology of many other epithelial cancers.

Thus the methods described above may be further understood in connection with the following additional examples.

Reagents. Resveratrol and pterostilbene were obtained from ChromaDex Inc. (Irvine, Calif.). Six week old female SKH-1 hairless mice were purchased from Charles River Labs (Willington, Mass.). Anti-CPD (clone TDM2) and anti-64 PP (clone 64M-2) antibodies purchased from CosmoBio USA (Carlsbad, Calif.).

UV-B exposure. Mice were exposed to a single dose of 180 mJ/cm² UVB radiation delivered via broadband UVB lamps (TL 40 W/12 RS; Philips) emitting 290-350 nm light with a peak emission at 312 nm. The irradiance was measured using a thermopile sensor (818P-001-12; Newport Inc., Irvine, Calif.) and power meter (842-PE; Newport Inc.).

ELISAS. DNA was isolated from snap frozen whole back skin using Qiagen QIAamp Blood mini kit (cat #51104) per manufacturer's instructions. ELISAs were performed on purified DNA with anti-CPD (clone TDM2) and anti-64 PP (clone 64M-2) antibodies using manufacturer's protocol and read using a DTX 800 Multimode Detector plate reader (Beckman Coulter) using standard settings from colorimetric detection. Four technical replicates were used and DNA-free wells were used as negative control.

Statistics. All statistical analyses were performed using CRAN R, version 3.0.0, an open-source program of The R Foundation for Statistical Computing. Linear mixed-effect models were fit for each endpoint with a random effect for the mouse and fixed effects for UV exposure, treatment group, and their interaction.

### EXAMPLE 1A

Pterostilbene treatment preventing harmful UV-B-mediated damage in the skin of SKH-1 mice.

Hairless SKH-1 mice were used to evaluate the effectiveness of pterostilbene at preventing UV-mediated damage in skin. The SKH-1 model is ideally suited for this purpose and is commonly used in preclinical studies and yielded valuable results. Previously, resveratrol has been demonstrated to prevent aberrant alterations in mouse skin (Reagan-Shaw, S., et al., 2004). To ascertain if pterostilbene could also prevent those molecular alterations, we replicated these experiments. Specifically, 15 adult female SKH-1 (hairless) mice were subjected to UV-B radiation (180 mJ/cm²) 30 minutes after topical application of vehicle (acetone), resveratrol or pterostilbene (5 mice each group), on their dorsal skin. For all topical treatments 200 μl total volume was applied to the back skin of the mouse. For acetone group, 200 μl of acetone was applied. For the resveratrol and pterostilbene groups the appropriate stilbene was applied at a concentration of 10 μmol/0.2 ml acetone/mouse. These mice were treated every 2 days for 7 total treatments. As a control, 15 mice were treated with topical vehicle, resveratrol or pterostilbene but not exposed to UV-B (5 mice each group). 24 hrs after last treatment, mice were euthanized, skin bi-fold measurements taken and back skins collected. As shown in FIGS. 2 and 3A-B, pterostilbene prevented UV-B-induced damage in mouse skin. Visually, pterostilbene clearly prevented redness (sunburn) on back skin as compared to vehicle+UV-B treated (FIG. 2; compare far right panel to far left panel). In fact, no redness was observed on any of the 5 mice in the pterostilbene+UV-B group. Further, while resveratrol was able to limit the amount of redness observed on the back, it did not prevent it like pterostilbene did (FIG. 2; compare far right panel to middle panel). This prevention of visual skin damage by pterostilbene correlated nicely with the prevention of DNA damage. Specifically, pterostilbene prevented cyclobutane pyrimidine dimer (CPD) and 64 pp formation (FIGS. 3A and 3B, respectively) as compared to vehicle+UV-B. In comparison, resveratrol only prevented 64 pp formation (FIG. 3B). Therefore pterostilbene prevented the 2 major types of UV-induced DNA damage (CPD and 64 pp) in the skin of SKH-1 mice. This is the first demonstration that pterostilbene was effective in the prevention of DNA damage. Interestingly, pterostilbene was more effective than resveratrol in all cases.

### EXAMPLE 1B

Pterostilbene treatment preventing both CPD and 64 pp DNA damage following UV-B exposure.

DNA was obtained from the back skin of the SKH-1 mice from the experiment above after completion of the 14-day experiment. This DNA was then examined for DNA damage (both CPD and 64 pp) quantitatively using ELISA as previously described (Nishiwaki, Y., et al., “Trichothiodystrophy fibroblasts are deficient in the repair of ultraviolet-induced cyclobutane pyrimidine dimers and (6-4) photoproducts,” J. Invest. Dermatol. (2004) 122: 526-32). A 96-well plate was coated with 0.003% protamine sulfate solution by adding 50 microliters of the solution per well and incubated overnight at 37° C. to dry. Wells were then washed with water and DNA from SKH-1 mice was denatured by heating to 100° C. for 10 minutes, rapidly cooled on ice and then added to each well at a concentration of 0.2 micrograms/ml for CPD ELISA or 4.0 micrograms/ml for the 64 pp ELISA. DNA from each mouse was examined in triplicate in each case. The plates were then allowed to dry completely at 37° C. overnight. ELISAs were then probed with either the Anti-CPD antibody (Cosmo Bio Co LTD Tokyo, Japan) or Anti-64 pp antibody (Cosmo Bio Co LTD) according to manufacturer protocols. Quantification of DNA damage was measured at 492 nm by a spectrophotometer. As shown in FIGS. 3A and 3B, pterostilbene prevented both CPD and 64 pp UV-mediated DNA damage. Specifically, while CPDs are quantitatively detected above background for both vehicle and resveratrol following UV-treatment, no CPDs were detected in the pterostilbene treated animals following UV-treatment (FIG. 3A). Similarly, while 64 pps are detected in the vehicle treated animals after UV exposure, no 64 pps are observed in the resveratrol or pterostilbene treated animals (FIG. 3B). Taken together, pterostilbene clearly prevented UV-mediated DNA damage (both CPD and 64 pp), while resveratrol only prevented UV-induced 64 pp formation.

### EXAMPLE 2

In another embodiment, pterostilbene treatment was shown to prevent harmful UV-B-mediated damage including hyperplasia in the skin of SKH-1 mice.

The protocol of Example 1A was repeated. As shown in FIGS. 2, 3A-B, and 4, pterostilbene prevented UV-B-induced damage in mouse skin. This prevention of visual skin damage by pterostilbene correlated nicely with the prevention of bi-fold skin thickening. To measure bi-fold skin thickness (mm), at least three measurements per mouse were taken using digital calipers on the dorsal skin. Specifically, pterostilbene prevented UV-mediated bi-fold skin thickening as compared to vehicle+UV-B (FIG. 3C). In comparison, resveratrol reduced the amount of bi-fold skin thickening, but did not prevent it (FIG. 3C). Furthermore, immunohistochemistry (H&E stain) of 10% formalin fixed paraffin embedded back tissue from these mice revealed that pterostilbene prevented UV-mediated hyperplasia at the cellular level (FIG. 4). Therefore pterostilbene prevented UV-induced hyperplasia of the skin of SKH-1 mice. This is the first demonstration that pterostilbene was effective in the prevention of hyperplasia. Interestingly, pterostilbene was more effective than resveratrol in all cases.

### EXAMPLE 3

In another embodiment, efficacy of oral pterostilbene in the prevention of UV-mediated SCC in SKH-1 mice can be demonstrated.

The above data clearly demonstrated that pterostilbene can prevent UV-mediated damage (including DNA damage) in the skin of SHK-1 mice. To determine if pterostilbene is efficacious in the prevention of SCC, the ability of pterostilbene to prevent UV-induced SCC in SKH-1 mice will be examined, SKH-1 mice will be used here as this model is particularly susceptible to UV-induced skin carcinogenesis (Dickinson S. E., et al., “p38 MAP kinase plays a functional role in UVB-Induced mouse skin carcinogenesis,” Mol. Carcinog. (2011) 50(6): 469-478). Eighty female SKH-1 mice (6 weeks old) will be obtained through the University of California—Irvine (UCI) mouse core facility and allowed to acclimate for 15 days prior to beginning experiments. Mice will be housed there in accordance with standards set by the Institutional Animal Care and Use Committee (IACUC) at UC Irvine. Mice will be allowed food and water ad libitum. Forty female SKH-1 mice (6 weeks old) will be randomly divided into two groups. The first group of animals will receive pterostilbene (in a 0.5% methylcellulose solution) by oral gavage three times a week (200 mg/Kg body weight). The second group of animals will receive oral vehicle (0.5% methylcellulose solution) by oral gavage and will serve as age-matched, untreated controls. Two weeks after first treatment mice will be exposed to UV irradiation using a solar simulator as previously described (Papazoglou, E., et al., “Noninvasive assessment of UV-induced skin damage: comparison of optical measurements to histology and MMP expression,” Photochem. Photobiol. (2010) 86: 138-45). UV treatments will be performed weekly for 25-weeks. The initial dose will be 0.9 kJ/m² UV-B and increased each week by 25% until the final dose is reached at 2.75 kJ/m² UV-B, which will be continued for the duration of the experiment. Skin tumors will be counted and measured using digital caliper weekly. At the conclusion of the 25 weeks mice test groups will be sacrificed. Back skins and tumors will be excised for further analysis. The time of tumor appearance, number and size of tumors as well as lifetime survival will be recorded for individual mice to evaluate the effectiveness of treatments compared to corresponding control group.

Evaluation of biomarkers in normal skin and SCC of SKH-1 mice. To examine the effect of pterostilbene on AA signaling in our model examples, changes in expression levels from normal skin and tumors from the control mice will be assessed and compared to normal skin and tumors from pterostilbene treated mice. Concentrations of 12-HETE and PGE₂ will be assessed in normal skin and tumors from the control mice and compared to normal skin and tumors from pterostilbene treated mice after the 25 week treatment period using liquid chromatography coupled to a triple quad mass spectrometer (LC/MS/MS) as previous described (Maskrey, B. H., et al. “Analysis of eicosanoids and related lipid mediators using mass spectrometry,” Biochem. Soc. Trans. (2008) 36: 1055-9). The concentrations of pterostilbene in mouse skin will also be assessed by LC/MS/MS as previously described (Kapetanovic, I. M., et al., 2011) to ensure effective delivery of pterostilbene to the skin. Protein levels of COX-2, 12-LOX, UGT and 15-PGDH will be measured semi-quantitatively by Western blot. DNA damage response proteins (e.g., p53 and H2AX) and angiogenesis (e.g., VEGF) will also be assessed in normal and tumor tissue. Detection of DNA damage (long term) will also be evaluated (both CPD and 64 pp).

The mouse models as described herein indicate that pterostilbene will be efficacious in the prevention of UV-mediated skin cancer. In addition, the data generated here supports the use of pterostilbene (e.g., applied topically) to treat, inhibit or prevent UV-mediated DNA damage (both CPD and 64 pp) in mammals, including human subjects. These data also support the use of pterostilbene (e.g., applied topically) to treat, inhibit or prevent hyperplasia in mammals, including human subjects.

In addition, these data provide support for the ability of pterostilbene to treat, inhibit or prevent a wide array of human cellular proliferative disorders including, but not limited to, benign hyperplasia (such as Psoriasis), keloid formation, AK formation, NMSC, as well as skin cancer (SCC).

Therefore, in other embodiments of the method, pterostilbene can be administered by any route of administration described herein (e.g., topical) to treat, inhibit or prevent proliferative disorders including, but not limited to, benign hyperplasia (such as Psoriasis), keloid formation, AK formation, NMSC, as well as skin cancer (SCC) in mammals, including humans.

When pterostilbene is used in a chemopreventive method, susceptible skin is treated prior to any visible condition affecting that skin (or, in some embodiments, any other evidence of DNA damage or hyperplasia) in a particular individual. When pterostilbene is used to inhibit DNA damage and/or hyperplasia, pterostilbene may be applied to skin that exhibits evidence of either of these conditions or skin that exhibits no such evidence. When pterostilbene is used to treat any of the conditions described above, pterostilbene may be applied to skin that exhibits evidence of one or more of these conditions. Treatment of all skin can be achieved by systemic administration.

Useful therapeutic dosages of pterostilbene can range, but are not limited to, from about 1 mg to about 1000 mg in a human individual. Another suitable dose range is from about 5 mg to about 500 mg. Another suitable dose range is from about 20 mg to about 250 mg. Pterostilbene may be formulated as a pharmaceutical or nutraceutical composition, including a pharmaceutically or nutraceutically acceptable carrier, respectively. In one embodiment of a pharmaceutical composition containing pterostilbene, a suitable level of pterostilbene may range from about 0.1% by weight to about 10% by weight, based on the total weight of the composition.

The cosmetic or cosmeceutical compositions of the present invention may be administered in combination with a nutraceutically acceptable carrier. The active ingredients in such formulations may comprise from 1% by weight to 99% by weight, or alternatively, 0.1% by weight to 99.9% by weight. Alternatively, the active ingredients can range from about 5% by weight to about 75% by weight, or from about 10% by weight to about 75% by weight. “Nutraceutically acceptable carrier” means any carrier, diluent or excipient that is compatible with the other ingredients of the formulation and not deleterious to the user. Useful excipients include microcrystalline cellulose, magnesium stearate, calcium stearate, any acceptable sugar (e.g., mannitol, xylitol), and for cosmetic use an oil-base is preferred.

In certain embodiments, the methods described herein reduce DNA damage, as indicated by formation of cyclobutane-pyrimidine dimers (CPDs) or 6-4 photoproducts (64 pps). In therapeutic methods, this reduction can be determined by measuring DNA damage before and after initiating treatment. In some embodiments, the methods described herein reduce hyperplasia, as indicated by bi-fold skin thickening. In therapeutic methods, this reduction can be determined by measuring bi-fold skin thickening before and after initiating treatment. Formation of CPDs or 6-4 64 pps or bi-fold skin thickening (e.g, of human subjects) can be measured by any suitable method, including those described herein.

The pharmaceutical (e.g., topical) compositions of the present invention may be administered in combination with a pharmaceutically acceptable carrier. The active ingredients in such formulations may comprise from 1% by weight to 99% by weight, or alternatively, 0.1% by weight to 99.9% by weight. “Pharmaceutically acceptable carrier” means any carrier, diluent or excipient that is compatible with the other ingredients of the formulation and not deleterious to the user.

In accordance with certain embodiments, the topical pharmaceutical compositions disclosed herein can be provided in the form of an ointment, cream, lotion, gel or other transdermal delivery systems as described in L. V. Allen, Jr., et al., Ansel's Pharmaceutical Dosage Forms and Drug Delivery Systems, 9^{th} Ed.,pp. 272-293 (Philadelphia, Pa.: Lippincott Williams & Wilkins, 2011) which is incorporated herein by reference.

Ointments, as used herein, refer to semi-solid preparations including an ointment base having one or more active ingredients incorporated or fused (i.e., melted together with other components of the formulation and cooled with constant stirring to form a congealed preparation) therein. The ointment base may be in the form of: an oleaginous or hydrocarbon base (e.g., petrolatum or a petrolatum/wax combination); an absorption base which permits the incorporation of aqueous solution resulting in the formation of a water-in-oil emulsion (e.g., hydrophilic petrolatum) or which is a water-in-oil emulsion that permits the incorporation of additional quantities of aqueous solutions (e.g., lanolin); a water-removable base which are oil-in-water emulsions that may be diluted with water or aqueous solutions (e.g., hydrophilic ointment, USP); or a water-soluble base that do not contain oleaginous components (e.g., polyethylene glycol (PEG) formulations which combine PEGs having an average molecular below 600 with a PEG having an average molecular weight above 1,000); and the like.

Creams, as used herein, refer to semisolid preparations containing one or more active or medicinal agent dissolved or dispersed in either a water-in-oil emulsion or an oil-in-water emulsion or in another type of water-washable base. Generally, creams are differentiated from ointments by the ease with which they are applied/spread onto a surface such as the skin and the ease with which they are removed from a treated surface.

Lotions, as used herein, refer to suspensions of solid materials in an aqueous vehicle. Generally, lotions have a non-greasy character and increased spreadability over large areas of the skin than ointments, creams, and gels.

Gels, as used herein, refer to semisolid systems including a dispersion of small and/or large molecules in an aqueous liquid vehicle which is rendered jellylike by the addition of a gelling agent. Suitable gelling agents include, but are not limited to, synthetic macromolecules (e.g., carbomer polymers), cellulose derivatives (e.g., carboxymethylcellulose and/or hydroxypropyl methylcellulose), and natural gums (e.g., tragacanth gum, carrageenan, and the like). Gel preparations may be in the form of a single-phase gel in which the active or medicinal ingredients are uniformly dispersed throughout the liquid vehicle without visible boundaries or a two-phase gel wherein flocculants or small distinct particles of the active or medicinal ingredient are dispersed within the liquid vehicle.

Transdermal preparations may be formed from an ointment, cream, or gel that has been combined with a penetration enhancer and are designed to deliver an active or medicinal ingredient systemically. Penetration enhancers include, for example, dimethyl sulfoxide, ethanol, propylene glycol, glycerin, PEG, urea, dimethyl acetamide, sodium lauryl sulfate, poloxamers, Spans, Tweens, lecithin, and/or terpenes amongst others.

Other suitable semi-solid forms for use as cosmetic and/or topical pharmaceutical compositions include pastes (preparations containing a larger proportion of solid material rendering them stiffer than ointments) and glycerogelatins (plastic masses containing gelatin, glycerin, water, and an active or medicinal ingredient).

In other embodiments the topical and/or cosmetic compositions can be prepared in accordance with dosage forms as described in Sample Preparation of Pharmaceutical Dosage Forms, B. Nickerson, Ed. (New York: Springer, 2011) herein incorporated by reference.

Useful daily topical dosages of pterostilbene can range, but are not limited to, from about 1 mg to about 1000 mg in a human individual. Another suitable daily topical dose range is from about 5 mg to about 500 mg. Another suitable daily topical dose range is from about 20 mg to about 250 mg. Pterostilbene can be provided in daily topical dosages of from about 10 mg to about 250 mg, in a human patient, for example. Another suitable topical dosage range is from about 50 mg to about 150 mg daily. Another suitable topical dosage range is from about 50 mg to about 100 mg daily. A particularly suitable dosage is about 100 mg administered daily.

For oral administration, pterostilbene may be combined with one or more solid inactive ingredients for the preparation of tablets, capsules, pills, powders, granules or other suitable dosage forms. For example, the active agent may be combined with at least one excipient such as fillers, binders, humectants, disintegrating agents, solution retarders, absorption accelerators, wetting agents, absorbents, or lubricating agents. Other useful excipients include magnesium stearate, calcium stearate, mannitol, xylitol, sweeteners, starch, carboxymethylcellulose, microcrystalline cellulose, silica, gelatin, silicon dioxide, and the like.

In some embodiments, compositions useful in the methods described herein can include, in addition to pterostilbene, another active agent useful for treating, inhibiting or preventing the unwanted effects of UV, such as UV-mediated DNA damage or hyperplasia. For example, pterostilbene can be administered in composition also containing any type of sunscreen, e.g., in topical and/or cosmetic compostions.

Routes of Administration

The compounds may be administered by any route, including but not limited to oral, sublingual, buccal, ocular, pulmonary, rectal, and parenteral administration, or as an oral or nasal spray (e.g. inhalation of nebulized vapors, droplets, or solid particles). Parenteral administration includes, for example, intravenous, intramuscular, intraarterial, intraperitoneal, intranasal, intravaginal, intravesical (e.g., to the bladder), intradermal, transdermal, topical, or subcutaneous administration. Also contemplated within the scope of the invention is the instillation of pterostilbene in the body of the patient in a controlled formulation, with systemic or local release of the drug to occur at a later time. For example, the drug may be localized in a depot for controlled release to the circulation, or for release to a local site of tumor growth.

The treatment may be carried out for as long a period as necessary, either in a single, uninterrupted session, or in discrete sessions. The treating physician will know how to increase, decrease, or interrupt treatment based on patient response. According to one embodiment, treatment is carried out for from about four to about twelve weeks. The treatment schedule may be repeated as required.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

1. A chemoprotective method for treating, inhibiting or preventing UV-mediated DNA damage or hyperplasia in an individual comprising administering to the individual in need of such treatment an effective amount of the compound pterostilbene.

2. The chemoprotective method of claim 1, wherein the individual is a human.

3. The chemoprotective method of claim 2, wherein the pterostilbene compound is provided in a composition comprising a pharmaceutically or nutraceutically acceptable carrier.

4. The chemoprotective method of claim 3, wherein the effective amount of pterostilbene for a total dose is in a range of about 0.1% by weight to about 10% by weight based on the total weight of the composition.

5. The chemoprotective method of claim 4, wherein the effective amount of pterostilbene for a total daily dose is in a range of about 1 mg to about 1000 mg.

6. The chemoprotective method of claim 1, wherein the route of administration of the compound is selected from the group consisting of oral, topical, intradermal, transdermal, and subcutaneous.

7. The chemoprotective method of claim 1, wherein DNA damage indicated by formation of cyclobutane-pyrimidine dimers (CPDs) or 6-4 photoproducts (64 pps) is decreased.

8. The chemoprotective method of claim 1, wherein hyperplasia indicated by bi-fold skin thickening is decreased.

9. A topical method for treating, inhibiting or preventing UV-mediated DNA damage in skin or hyperplasia in an individual comprising administering to the individual in need of such treatment an effective amount of the compound pterostilbene to the skin surface.

10. The topical method of claim 9, wherein the individual is a human.

11. The topical method of claim 10, wherein the pterostilbene compound is provided in a composition comprising a pharmaceutically acceptable carrier.

12. The topical method of claim 11, wherein the effective amount of pterostilbene for a total dose is in a range of about 0.1% by weight to about 10% by weight based on the total weight of the composition.

13. The topical method of claim 12, wherein the effective amount of pterostilbene for a total daily dose is in a range of about 1 mg to about 1000 mg.

14. The topical method of claim 9, wherein DNA damage in skin indicated by formation of cyclobutane-pyrimidine dimers (CPDs) or 6-4 photoproducts (64 pps) is decreased.

15. The topical method of claim 9, wherein hyperplasia indicated by bi-fold skin thickening is decreased.
